(19) United States
(12) Patent Application Publication
(10) Pub. No.: US 2024/0331853 A1
(43) Pub. Date: Oct. 3, 2024

(54) SYSTEMS AND METHODS TO DETERMINE A LIKELIHOOD OF IMAGING WORKFLOW DELAYS

(71) Applicant: KONINKLIJKE PHILIPS N.V., EINDHOVEN (NL)

(72) Inventors: Olga STAROBINETS, NEWTON, MA (US); Ekin KOKER, CAMBRIDGE, MA (US); Siva Chaitanya CHADUVULA, MALDEN, MA (US); Ranjith Naveen TELLIS, TEWKSBURY, MA (US); Sandeep Madhukar DALAL, WINCHESTER, MA (US); Yuechen QIAN, LEXINGTON, MA (US)

(21) Appl. No.: 18/619,344

(22) Filed: Mar. 28, 2024

Related U.S. Application Data
(60) Provisional application No. 63/455,634, filed on Mar. 30, 2023.

(51) Int. Cl. G16H 40/20; G16H 10/60; G16H 40/67

(52) U.S. Cl. CPC G16H 40/20; G16H 10/60; G16H 40/67

(57) ABSTRACT

A non-transitory computer readable medium (26s) stores instructions executable by at least one electronic processor (14s) to perform a method (100) of preparing for an upcoming medical imaging examination. The method includes receiving information about the upcoming medical imaging examination; receiving patient data from a patient monitoring device (40) associated with a patient who is to undergo the upcoming medical imaging examination; analyzing the received patient data based on information about the upcoming medical imaging examination; determine one or more indicators of a potential issue with the upcoming medical imaging examination based on the comparison of the received patient data with the information about the upcoming medical imaging examination; and outputting a warning (46) indicative of the determined one or more indicators of the issue with the medical imaging examination.

This application claims the benefit of U.S. Provisional Patent Application No. 63/455,634 filed Mar. 30, 2023. This application is hereby incorporated by reference herein.

The following relates generally to the medical imaging arts, patient preparation arts, remote imaging assistance arts, remote imaging examination monitoring arts, patient data monitoring arts, imaging workflow monitoring arts, and related arts.

### BACKGROUND

There are ongoing efforts to improve the efficiency, accuracy, and clinical value of radiology examinations such as magnetic resonance imaging (MRI) examinations, computed tomography (CT) examinations, positron emission tomography (PET) examinations, and so forth. As one example, radiology operations command centers (ROCC) aim to improve the way radiology services are offered by expanding access to imaging across the organization, increasing patient and staff satisfaction, improving patient outcomes and lowering the cost of care. This is achieved by enabling virtualized imaging operations (connecting expert users to a local technologist, providing tools for guidance and over the shoulder support), through workflow orchestration (by enabling intelligent scheduling and predicting and proactively addressing sources of potential bottlenecks), through protocol standardization, etc.

While approaches such as ROCCs provide substantial performance improvement during an imaging examination, such approaches do not account for factors outside of the context of the actual imaging examination. These outside factors can determine the success (or failure) of an imaging examination, but are typically outside of the control of the local technologist and cannot be remediated by ROCC access to an expert user. Successful medical imaging examinations rely on accounting for any patient-specific limitations such as mobility limitations or presence of an implanted pacemaker, and on patient compliance with any pre-examination preparation instructions such as fasting 24 hours ahead of the examination. In an attempt to control these outside factors, patients typically are asked to complete a pre-examination questionnaire to identify any patient-specific limitations, and are provided with any pre-examination preparation instructions. However, patients may forget to disclose their limitations, or in some instances may fail to do so out of embarrassment in the case of conditions that are sometimes socially stigmatized, such as obesity. Patients may also fail to comply with pre-examination preparatory instructions, and may fail to report such non-compliance out of embarrassment or failure to appreciate the impact the noncompliance may have on the medical imaging examination. Even if these situations do not occur, an imaging examination may fail or be problematic due to unexpected patient issues such as extreme nervousness, over-exertion, or the like. These types of issues can result in delays in the examination or to being unable to complete the examination leading to rescheduling which can be costly to both the patient and the radiology department.

The following discloses certain improvements to overcome these problems and others.

### SUMMARY

In one aspect, a non-transitory computer readable medium stores instructions executable by at least one electronic processor to perform a method of preparing for an upcoming medical imaging examination. The method includes receiving information about the upcoming medical imaging examination; receiving patient data from a patient monitoring device associated with a patient who is to undergo the upcoming medical imaging examination; analyzing the received patient data to determine one or more indicators of a potential issue with the upcoming medical imaging examination based on a comparison of the received patient data with the information about the upcoming medical imaging examination; and outputting a warning indicative of the determined one or more indicators of the issue with the medical imaging examination.

In another aspect, a system is disclosed for preparing for an upcoming medical examination. The system comprises: a patient monitoring device associated with a patient who is to undergo the upcoming medical imaging examination; and at least one electronic processor programmed to perform a method of preparing for the upcoming medical imaging examination. The method includes receiving information about the upcoming medical imaging examination; providing a user interface (UI) on the patient monitoring device; receiving an input on the UI indicative of a consent of the patient to transmit patient data from the patient monitoring device; analyzing the received patient data based on the information about the upcoming medical imaging examination to determine one or more indicators of a potential issue with the upcoming medical imaging examination; and outputting a warning indicative of the determined one or more indicators of the issue with the medical imaging examination.

In another aspect, a method of preparing for an upcoming medical imaging examination includes acquiring patient data using a patient monitoring device connected with a patient who is to undergo the upcoming medical imaging examination; transmitting the patient data to a medical information technology (IT) system; using the medical IT system, analyzing the patient data using a context of the upcoming medical imaging examination stored at the medical IT system to determine one or more indicators of a potential issue with the upcoming medical imaging examination; and outputting, on a user interface operatively connected with the medical IT system, a warning indicative of the determined one or more indicators of the issue with the medical imaging examination.

One advantage resides in determining a potential delay in imaging workflows.

Another advantage resides in monitoring patient behavior before a medical imaging examination to predict potential delays in imaging workflows.

Another advantage resides in preventing delays in imaging workflows due to patient behavior.

Another advantage resides in reducing costs and delays in imaging workflows.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative system for providing remote assistance in accordance with the present disclosure.

FIG. 2 shows an example flow chart of operations suitably performed by the system of FIG. 1.

FIG. 3 illustrates a nonlimiting illustrative example of a consent form displayed on a patient monitoring device via which the patient can select type(s) of patient data to collect and send to a radiology department for use in pre-examination preparatory patient monitoring, and a time period over which that data is agreed to be collected.

FIG. 4 diagrammatically shows a system for monitoring a patient prior to an upcoming imaging examination to identify and recommend action items to improve the likelihood of success of the upcoming imaging examination.

### DETAILED DESCRIPTION

Smart devices are quickly becoming ubiquitous. Many people own a smart phone, which can provide various vital sign monitoring capabilities on its own (e.g., pedometer functionality utilizing the built-in accelerometer, or pulse rate monitoring utilizing the built-in camera) or by wired or wireless connection with dedicated vital sign monitors. Medical monitors such as electrocardiogram (EKG) monitors, electroencephalography (EEG) headbands or skullcaps, wireless blood pressure monitors, continuous positive airway pressure (CPAP) machines that collect patient sleep data, and so forth are sometimes prescribed by a patient's physician. Still further, consumer grade health and wellness monitors are becoming more popular, such as fitness monitors (e.g., FitBit®, Bellabeat Leaf®), digestive trackers (e.g. FoodMarble®), and baby monitors as some examples. Such medically prescribed and/or personal electronic devices can produce massive amounts of patient data. It is recognized herein that such data collected the day before or on the day of a medical procedure or appointment could be used to predict potential complications that may arise during the medical exam-such as a radiology (i.e. medical imaging) examination.

However, there are substantial challenges to leveraging the patient data collected by these devices for controlling outside factors in order to improve the quality of medical imaging examinations. Prescribed medical monitors (EKG, ECG, blood pressure, CPAP, et cetera) and consumer grade personal electronic devices (cell phones, fitness monitors, digestive trackers, et cetera) are not designed to provide pre-examination monitoring to assist in medical imaging examinations. The relevance of the collected patient data to a medical imaging examination is not readily apparent, and moreover the relevance can vary substantially from one patient to the next and from one imaging examination to the next. For example, eating a meal 6 hours before an imaging examination is of no consequence for some types of medical imaging examinations, but can render other types of examinations unreliable. Similarly, intensive physical activity shortly before an imaging examination may be irrelevant to the examination, but for certain types of imaging examinations such as metabolic studies, intensive pre-examination physical activity can degrade the clinical value of the results of the imaging examination. Furthermore, the radiology department usually does not have access to the patient data collected by a prescribed wearable patient monitor, much less patient data generated by the patient's personal electronic devices.

The following discloses approaches for overcoming such challenges and leveraging a patient monitoring device or devices to identify or predict outside factors that may impact an upcoming medical imaging examination. The monitoring devices may be smartphones loaded with a special application program (“app”), a personal health tracker such as a Fitbit®, a medical monitoring device such as a mobile electrocardiogramonitor or a brain-sensing headband, a personal breath analyzer such as a FoodMarble®, or some combination thereof. With patient consent provided for a defined time period before the imaging examination, the patient data collected by the patient monitoring device(s) over the defined time period before the imaging examination (e.g., 1-2 days as an example) are transmitted via the Internet to a computing system (e.g., on-premise server, cloud computing resource, etc.) accessible by or in connection with the radiology department. For example, such a computing system may be part of a Picture Archiving Communication System (PACS), Radiology Information System (RIS), ROCC, or similar. In some embodiments, patient data is automatically deleted after the imaging examination is complete, or after the corresponding radiology report is filed.

The computing system also receives examination context information such as information about the upcoming medical imaging examination (e.g., fasting requirements), patient demographic information, and any available information on chronic conditions of the patient. The computing system analyzes the patient information in view of the examination context to determine actionable information relating to the upcoming imaging examination (including the imaging data acquisition, the subsequent interpretation of acquired patient images by a radiologist, or both).

As one example, analysis of Fitbit® data or pedometer data acquired by an app loaded on the patient's cellphone can indicate the patient has mobility issues. This constitutes actionable information on the basis of which the radiology department can ensure that a wheelchair or other patient transport and additional staff are available to assist the patient.

As another example, analysis of the patient data from a breath analyzer can indicate that the patient has consumed food during a fasting interval preceding the imaging examination. This constitutes actionable information on the basis of which the imaging examination may be rescheduled if the fast was essential, or this actionable information may be conveyed to the radiologist who interprets the imaging examination to ensure the radiologist takes into account the fasting violation when interpreting the images.

As another example, analysis of patient data from a mobile ECG may detect possible presence of an implanted pacemaker by detecting an apparently regulated patient's heart rate. This constitutes actionable information for an upcoming magnetic resonance imaging (MRI) examination on the basis of which the radiology department can investigate to confirm the patient actually has a pacemaker and, if so, whether it is MRI-compliant.

As another example, analysis of cardiac and/or blood pressure data from a Fitbit® or mobile ECG monitor or the like can detect patient anxiety about the upcoming imaging examination as frequent periods of elevated heart rate. This constitutes actionable information on the basis of which an annotation may be made in the imaging schedule that the patient may be experiencing anxiety, and/or the patient may be prescribed a pre-exam sedative.

As another example, analysis of Fitbit® or similar patient data can detect patient over-exertion. In the context of a metabolic imaging examination, this constitutes actionable information on the basis of which the patient may be instructed to rest ahead of the examination or, in some cases, the metabolic imaging examination may need to be rescheduled.

As yet another example, similar detection of a cardiac arrhythmia or other irregular cardiac rhythm can constitute actionable information in the context of a cardiac imaging examination that is annotated to the patient's imaging examination for consideration by the radiologist in interpreting the cardiac imaging examination.

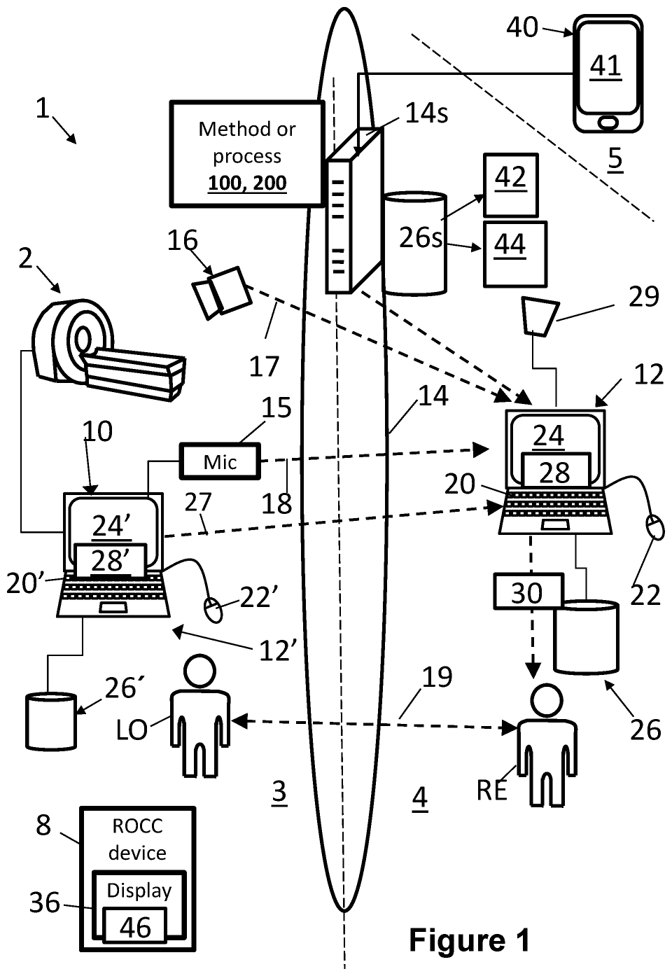

In the following, a non-limiting illustrative example of a setting in which the disclosed approaches for preparing for an upcoming medical imaging examination may be employed is described. The illustrative setting employs a Radiology Operations Command Center (ROCC) described below with reference to FIG. 1 to provide in-examination assistance from a remote expert to a local operator who is performing a medical imaging examination. The disclosed pre-examination preparatory assistance is usefully combined with such the ROCC to improve efficacy of the medical imaging examination both before and during the examination. However, it will be appreciated that the disclosed approaches for preparing for an upcoming medical imaging examination may be employed in conjunction with medical imaging examinations generally, including examinations that are performed without availability or assistance of the ROCC.

With reference to FIG. 1, a ROCC system 1 for providing assistance from a remote medical imaging expert RE (shortened to “remote expert” hereinafter, but sometimes also known as a “supertech”) to a local technologist operator LO is shown. As shown in FIG. 1, the local operator LO, who operates an imaging device (also sometimes referred to as an image acquisition device, medical imaging device, and so forth in the art) 2, is located in an imaging device bay 3, and the remote expert RE is disposed in a remote service location or center (hereinafter “remote location”) 4. It should be noted that the remote expert RE may not necessarily directly operate the imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the imaging device bay 3 (this may be, for example, in the case of a “remote operator or expert” RE who is a radiologist tasked with peri-examination image review), but more typically the remote location 4 and the imaging device bay 3 are in different buildings, and indeed may be located in different cities, different countries, and/or different continents. In general, the remote location 4 is remote from the imaging device bay 3 in the sense that the remote expert RE cannot directly visually observe the imaging device 2 in the imaging device bay 3 (hence optionally providing a video feed as described further herein).

The imaging device 2 can be a Magnetic Resonance (MR) imaging device, a Computed Tomography (CT) imaging device; a positron emission tomography (PET) imaging device; a single photon emission computed tomography (SPECT) imaging device; an X-ray imaging device; an ultrasound (US) imaging device; or an imaging device of another modality. The imaging device 2 may also be a hybrid imaging device such as a PET/CT or SPECT/CT imaging system. While a single imaging device 2 is shown by way of illustration in FIG. 1, more typically a medical imaging laboratory will have multiple imaging devices, which may be of the same and/or different imaging modalities. For example, if a hospital performs many CT imaging examinations and relatively fewer MRI examinations and still fewer PET examinations, then the hospital's imaging laboratory (sometimes called the “radiology lab” or some other similar nomenclature) may have three CT scanners, two MRI scanners, and only a single PET scanner. This is merely an example. Moreover, the remote location 4 may provide service to multiple hospitals. The local operator controls the imaging device 2 via an imaging device controller 10. The remote operator is stationed at a remote electronic processing device 12 (or, more generally, an electronic controller 12).

As diagrammatically shown in FIG. 1, in some embodiments, a camera 16 (e.g., a video camera) is arranged to acquire a video stream or feed 17 of a portion of a workspace of the imaging device bay 3 that includes at least the area of the imaging device 2 where the local operator LO interacts with the patient, and optionally may further include the imaging device controller 10. In some examples, the camera 16 is disposed in the imaging device bay 3, or disposed in a control room (not shown) where the local operator LO is situated to perform the medical examination. In other embodiments, a microphone 15 is arranged to acquire an audio stream or feed 18 of the workspace that includes audio noises occurring within the imaging device bay 3 (e.g., verbal instructions by the local operator LO, questions from the patient, and so forth). The video stream 17 and/or the audio stream 18 is sent to the remote electronic processing device 12 via the communication link 14, e.g., as a streaming video feed received via a secure Internet link.

The communication link 14 also provides a natural language communication pathway 19 for verbal and/or textual communication between the local operator and the remote operator. For example, the natural language communication pathway 19 may be a Voice-Over-Internet-Protocol (VOIP) telephonic connection, an online video chat link, a computerized instant messaging service, or so forth. Alternatively, the natural language communication pathway 19 may be provided by a dedicated communication link that is separate from the communication link 14 providing the data communications 17, 18, e.g., the natural language communication pathway 19 may be provided via a landline telephone. In some embodiments, the natural language communication link 19 allows a local operator LO to call a selected remote expert RE. The call, as used herein, can refer to an audio call (e.g., a telephone call), a video call, or an audio-video call. In another example, the natural language communication pathway 19 may be provided via a ROCC device 8 with a display device 36. For example, an “app” can run on the ROCC device 8 (operable by the local operator LO) and the remote electronic processing device 12 (operable by the remote expert RE) to allow communication (e.g., audio chats, video chats, and so forth) between the local operator LO and the remote expert RE.

FIG. 1 also shows, in the remote location 4 including the remote electronic processing device 12, such as a workstation, a workstation computer, or more generally a computer, which is operatively connected to receive and present the video feed 17 of the imaging device bay 3 from the camera 16 and/or to the audio feed 18. Additionally or alternatively, the remote workstation 12 can be embodied as a server computer or a plurality of server computers, e.g., interconnected to form a server cluster, cloud computing resource, or so forth. The workstation 12 includes typical components, such as an electronic processor 20 (e.g., a microprocessor), at least one user input device (e.g., a mouse, a keyboard, a trackball, and/or the like) 22, and at least one display device 24 (e.g., an LCD display, plasma display, cathode ray tube display, and/or so forth). In some embodiments, the display device 24 can be a separate component from the remote electronic processing device 12. The display device 24 may also comprise two or more display devices. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24. The video feed 17 from the camera 16 can also be displayed on the display device 24, and the audio feed 18 can be output on the remote electronic processing device 12 via a loudspeaker 29. In some examples, the audio feed 18 can be an audio component of an audio/video feed (such as, for example, outputting the audio feed 18 as a video cassette recorder (VCR) device would operate.

FIG. 1 shows an illustrative local operator LO, and an illustrative remote expert RE (e.g., supertech). However, in a Radiology Operations Command Center (ROCC) as contemplated herein, the ROCC provides a staff of supertechs who are available to assist local operators LO at different hospitals, radiology labs, or the like. Each remote expert RE can operate a corresponding remote electronic processing device 12. The ROCC may be housed in a single physical location or may be geographically distributed. For example, in one contemplated implementation, the remote expert RE are recruited from across the United States and/or internationally in order to provide a staff of supertechs with a wide range of expertise in various imaging modalities and in various imaging procedures targeting various imaged anatomies. A server computer 14s can be in communication with the medical imaging bay 3 and the remote location 4 with one or more non-transitory storage media 26s. The non-transitory storage media 26s may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the server computer 14s, various combinations thereof, or so forth. It is to be understood that any reference to a non-transitory medium or media 26s herein is to be broadly construed as encompassing a single medium or multiple media of the same or different types. Likewise, the server computer 14s may be embodied as a single electronic processor or as two or more electronic processors. The non-transitory storage media 26s stores instructions executable by the server computer 14s.

The imaging device controller 10 in the imaging device bay 3 also includes similar components as the remote electronic processing device 12 disposed in the remote location 4. Except as otherwise indicated herein, features of the imaging device controller 10, which includes a local workstation 12′, disposed in the imaging device bay 3 similar to those of the remote workstation 12 disposed in the remote location 4 have a common reference number followed by a “prime” symbol, and the description of the components of the imaging device controller 10 will not be repeated. In particular, the imaging device controller 10 is configured to display a GUI 28′ on a display device or controller display 24′ that presents information pertaining to the control of the imaging device 2, such as configuration displays for adjusting configuration settings, an alert 30 perceptible at the remote location when the status information on the medical imaging examination satisfies an alert criterion of the imaging device 2, imaging acquisition monitoring information, presentation of acquired medical images, and so forth. A screen mirroring data stream 27 carries the content presented on the display device 24′ of the imaging device controller 10. The screen mirroring data stream 27 can be acquired in various ways. In one approach, the screen mirroring data stream 27 can be acquired by screen mirroring software running on the imaging device controller 10. In another approach, screen scraping hardware is interposed between the display device 24′ of the imaging device controller 10 and the at least one electronic processor 20′ (e.g., video card or the like) that outputs the video signal. In this later approach, the screen scraping hardware may, by way of nonlimiting illustrative example, include a video splitter that splits the video signal, with one video signal being sent to the display device 24′ and the other being sent to the remote electronic processing device 12 as the screen mirroring data stream 27. The communication link 14 allows for screen sharing between the display device 24 in the remote location 4 and the display device 24′ in the imaging device bay 3. The GUI 28′ includes one or more dialog screens, including, for example, an examination/scan selection dialog screen, a scan settings dialog screen, an acquisition monitoring dialog screen, among others. The GUI 28′ can be included in the video feed 17 and displayed on the remote workstation display 24 at the remote location 4.

The foregoing components relate to the ROCC system 1, and advantageously provide for assisting the local operator LO during the imaging examination. Additionally, approaches are disclosed herein for preparing for the upcoming medical imaging examination before it is commenced. These approaches may be employed with or without the illustrative ROCC system 1.

To implement the pre-examination preparation assistance, FIG. 1 also shows a patient monitoring device 40 located in a space diagrammatically denoted as space 5 in FIG. 1. The space 5 diagrammatically represents wherever the patient is in a defined time period (e.g. one to two days, in one nonlimiting illustrative example) prior to the upcoming medical imaging examination. For example, the space 5 may represent the patient's home, workplace, workout gym, church or other religious house, various combinations thereof, and/or so forth where the patient may be over the course of the time period before the imaging examination. Typically, the space 5 will not include the imaging bay 3 or the remote location 4 (although in some cases the space 5 might include the hospital if the patient goes for a pre-examination appointment). The patient monitoring device 40 is worn by the patient (e.g., in the case of the device 40 being a worn EKG monitor) or carried by the patient (e.g. in the case of the device 40 being a smartphone) or is otherwise proximate to the patient (e.g. at night in the case of the device 40 being a CPAP) during at least a portion of the time period before the imaging examination. While a single device 40 is shown, it will be appreciated that the patient may carry or interact with two or more such patient monitoring devices, and/or that multiple patient monitoring devices may operate in combination as a single operative unit (e.g. a health/wellness tracking device that is linked with the patient's smartphone, or a medical device with multiple types of monitoring or detection capabilities).

The patient monitoring device 40 is configured to acquire patient data of the patient who is to undergo the medical imaging examination, and to wirelessly transmit the patient data to the server 14s, e.g. via cell tower, Wi-Fi, or the like. As shown in FIG. 1, the patient monitoring device 40 comprises a cell phone of the patient, but the patient monitoring device 40 can comprise any suitable smart device (or combination of smart devices) that collects patient data from the patient (e.g., a mobile EKG monitor, a Bellabeat Leaf device, a FoodMarble device, a brain sensing headband, a Fitbit, an Apple Watch, and so forth). Such patient data can include fitness tracking data, respiratory tracking data, heart rate data, sleep data, weight data, diet data, EKG data, EEG data, and so forth of the patient.

The server 14s also stores, in the non-transitory storage media 26s, an imaging protocols repository 42 of a medical facility that stores a plurality of imaging protocols to be used in medical imaging examinations. In some examples, the imaging protocols repository 42 includes imaging protocols for a plurality of medical facilities.

Furthermore, as disclosed herein, the server 14s performs a method or process 100 for preparing for an upcoming medical imaging examination of a patient performed using an imaging device 2 (i.e., by assisting a local operator LO of the imaging device 2 during medical imaging examinations by a remote expert RE). By preparing for an upcoming medical imaging examination using collected patient data, the method or process 100 may advantageously homogenize (i.e., uniformly) and predict potential delays in the workflow and suggest remedying measures to prevent such potential delays.

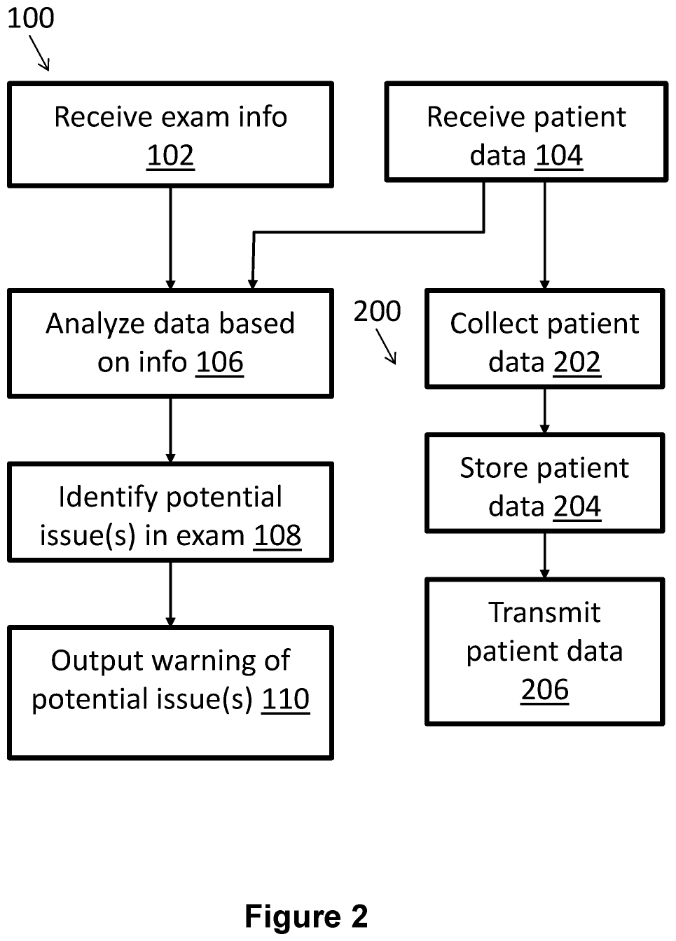

With reference to FIG. 2, and with continuing reference to FIG. 1, an illustrative embodiment of the method 100 is diagrammatically shown as a flowchart. To begin the method 100, at an operation 102, information about the upcoming medical imaging examination is received by the server computer 14s. The medical imaging examination information can include, for example, one or more of fasting requirements, patient demographic information, and any available information on chronic conditions of the patient. This information can be retrieved from the imaging protocols repository 42, for example.

At an operation 104, patient data associated with the patient is received from the patient monitoring device 40. The received patient data can include, for example, one or more of a presence of an implant in the patient, a use of a walking assistance device by the patient, and one or more vital signs of the patient (e.g., fitness tracking data, respiratory tracking data, heart rate data, sleep data, weight data, diet data, EKG data, EEG data, and so forth). It will be appreciated that the operations 102 and 104 can be performed concurrently or sequentially in either order.

In some embodiments, the patient data operation 104 includes receiving a user input indicative of the patient providing consent for collecting the patient data from the patient monitoring device 40. In such examples, conditional on the patient providing the consent, a wireless data connection from the patient monitoring device 40 to a medical information technology (IT) system 44 over which connection the patient data is received. The medical IT system 44 can be implemented in the server computer 14s or a different sever computer. In some examples, a user interface (UI) 41 is provided on the patient monitoring device 40 via which the patient selects which data to share with the medical IT system 44.

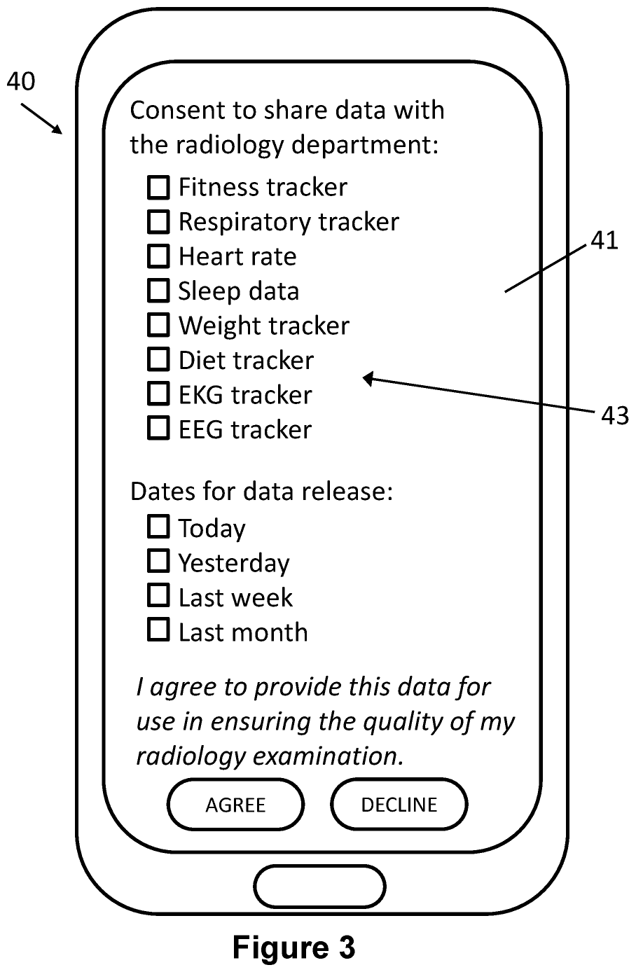

With brief reference to FIG. 3, a nonlimiting illustrative example of a consent form 43 that suitably is displayed on the display 41 of the patient monitoring device 40 (here a cellphone 40) is shown. In this example, the cellphone is programmed (e.g. by a fitness app) and/or operatively connected with ancillary patient monitoring devices to collect patient data of various types listed in the consent form 43, each with a selection box to the left of the item that the user can check or uncheck to indicate which data source(s) the patient wants to send to the radiology department. The lower portion of the consent form 43 provides for selection of the defined time period over which the patient consents for this data to be sent. In illustrative FIG. 3 the patient has checked (i.e. selected) the time period including yesterday and today, but has not selected (i.e. has left unchecked) the earlier time periods of last week and last month. The consent form 43 includes “AGREE” and “DECLINE” buttons by which the patient can agree to provide the data, or decline to do so, respectively. Assuming the patient selects “AGREE”, the patient monitoring device 40 will then commence (subject to wireless connectivity) to transmit yesterday's patient data and will continue to transmit today's patient data as it becomes available over the day.

In another embodiment, the server computer 14s stores executable instructions for performing an operation method 200 for operating the patient monitoring device 40. The operation method 200 includes, at an operation 202, collecting the patient data using the patient monitoring device 40. At an operation 204, the patient data is stored on the patient monitoring device 40, and/or stored to an electronic medical record of the patient. At an operation 206, the patient data is transmitted to the medical IT system 44.

At an operation 106, the received patient data (from the patient data operation 104) is analyzed based on information about the upcoming medical imaging examination (from the operation 102). At an operation 108, one or more indicators of a potential issue with the upcoming medical imaging examination can be determined based on the comparison of the received patient data with the information about the upcoming medical imaging examination. At an operation 110, a warning 46 indicative of the determined one or more indicators of the issue with the medical imaging examination can be output on the display device 36 of the ROCC device 8 for visualization by the local operator LO.

In one particular example, the patient data includes at least pedometer data indicative of a mobility status of the patient, and the warning 46 includes an indication that a wheelchair or other walking assistance device is available for the patient for the medical imaging examination.

In another particular example, the patient data includes at least patient breath data indicative of whether the patient has consumed food during a require fasting period before the medical imaging examination, and the warning 46 includes an indication that a radiologist should account for the food consumption by the patient.

In another particular example, the patient data includes at least a presence of an implant in the patient, and the warning 46 includes an indication that a radiologist should account for the implant presence during the medical imaging examination.

In another particular example, the patient data includes one or more of cardiac and blood pressure data of the patient, and the warning 46 includes an indication to determine whether the patient should be administered a sedative prior to the medical imaging examination.

In another particular example, the patient data includes patient over-exertion, and the warning 46 includes an indication to determine whether the patient should rest prior to the medical imaging examination or whether to reschedule the medical imaging examination.

In another particular example, the patient data includes cardiac data of the patient, and the warning 46 includes an indication to account for a cardiac arrhythmia or other irregular cardiac rhythm during interpretation of the medical imaging examination.

These are merely illustrative examples, and should not be construed as limiting.

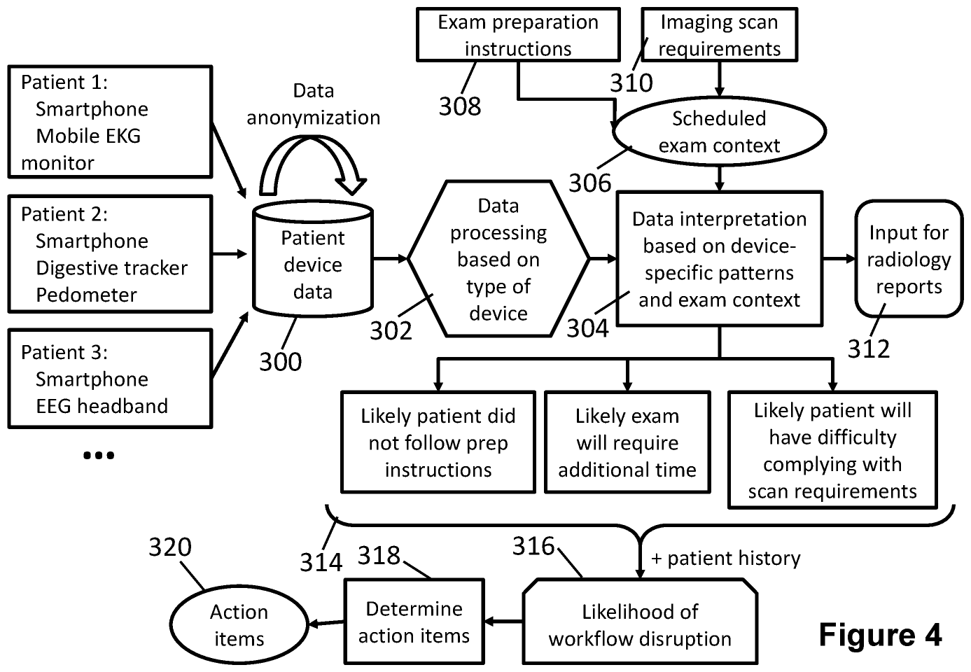

With reference now to FIG. 4, a further embodiment is illustrated of a system for monitoring a patient prior to an upcoming imaging examination to identify and recommend action items to improve the likelihood of success of the upcoming imaging examination. A patient device database 300 collects patient data for an illustrative three patients via their respective patient monitoring devices. Specifically, a Patient 1 has patient monitoring devices including a smartphone and a mobile EKG monitor. Patient 2 has patient monitoring devices including a smartphone, a digestive tracker (e.g. a FoodMarble®), and a pedometer. Patient 3 has patient monitoring devices including a smartphone and an EEG headband. These are merely nonlimiting illustrative examples. The patient database 300 includes or performs suitable data anonymization as previously described. In an operation 302 suitably performed by the server 14s, data processing is performed on the patient data contained in the patient database 300 to identify anomalies or patterns of interest that may impact the upcoming imaging examination. In an operation 304, these anomalies and/or patterns of interest are interpreted based on a context 306 of the upcoming imaging examination. The context 306 can include, for example, examination preparation instructions 308 and/or imaging scan requirements 310. The information 308, 310 may be extracted from a radiology examination card, radiology examination order, or the like stored in the radiology department IT system.

The output of the context-informed patient data interpretation 304 may serve as input 312 to radiology reports. For example, anomalous EKG activity recorded prior to the imaging examination and detected via the operations 302 and 304 may be provided at a radiology workstation as the radiologist is preparing a reading of the completed radiology examination images. The output of the context-informed patient data interpretation 304 may also serve as the basis for various likelihood determinations 314, such as a likelihood that the patient failed to follow the examination preparation instructions (e.g., if the digestive tracker detects food consumption within a 12 hour fasting period for an upcoming imaging examination), and/or a likelihood that the examination will require additional time (e.g., if the patient data indicates the patient is frail), and/or a likelihood that the patient will have difficulty complying with scan requirements (e.g., if the patient data indicates the patient has difficulty breathing and thus may be unable to comply with a breath-hold requirement during the upcoming imaging examination), and/or so forth. These likelihoods 314 along with any auxiliary information such as patient history data from a patent Electronic Medical Record (EMR) is combined to estimate an overall likelihood 316 of imaging examination workflow disruption, and to determine 318 action items 320 that may be taken to mitigate such disruption. The action items 320 could, for example, include increasing the time block for the imaging examination (e.g. if the exam is likely to require additional time), moving the examination to a different imaging device (e.g., a large-bore CT scanner if the patient data indicates the patient is obese), scheduling a senior imaging technologist to handle the upcoming imaging examination (e.g. if the patient is frail); or, in more extreme cases, rescheduling the imaging examination (e.g., if the patient has not followed a pre-examination fasting instruction that was essential to obtaining a clinically useful imaging examination.

### EXAMPLES

The foregoing are merely nonlimiting illustrative examples. The patient data collected by the patient monitoring device 40 can be collected on the day prior/the day of the imaging exam to identify patients who are more likely to have a longer/more challenging exams, such as identifying patients who may have difficulties following exam instructions (i.e. breath holds), patients who are anxious about the exam, patients who may need to be rescheduled-patients who have not complied with preparation instructions (i.e. fasting), etc.

To do so, a patient connects the patient monitoring device(s) 40 through some type of an app and allows for the patient data to be collected within a limited time window. The data is sent to a repository on the server computer 14s. A data processing module implemented in the server computer 14s processes the patient data based on its source, identifying anomalies or patterns of interest. An interpretation module implemented in the server computer 14s data is configured to accept inputs, process the inputs, combining them with information regarding the scheduled exam. The likelihood of a patient potential non-compliance, difficulty, anxiety is combined with the patient's past history of absenteeism, exam difficulties, etc. to produce a likelihood that patient's visit will result in a workflow disruption. Once the likelihood of workflow disruption is determined, appropriate proactive steps to address potential patient difficulties are recommended.

Analyzing data that is being routinely collected by personal patient monitoring device(s) 40 on the day of (or the day prior) to an appointment can help identify patients that are non-compliant with prep instruction, anxious, or exhibit factors that can potentially derail or delay exams. Identifying such patients could allow for proactive measures to be taken to resolve some of the patient difficulties and ensure an appropriate amount of time is allotted within the workflow.

There is a wide range of personal health/behavior data that is being routinely collected by personal health and other devices such as smart phones, smart watches, wristbands, headbands, mobile monitors, etc.

To perform the preparing method 100, patient data collection can take place through an app installed on a patient's monitoring device 40, enabling data to be transferred to the server computer 14s for processing. The data collection will be limited in time and scope and can involve an anonymization scheme to protect user privacy. The patient can be incentivized to share the patient monitoring device 40 data by offering discounts, transportation, parking perks, etc.

The anonymized patient data can be tagged with its device source and processed accordingly. A patient health profile will be created by processing raw patient data when necessary and accumulating feedback from personal patient monitoring devices 40 when available. Certain devices provide a layer of processing that does not need to be replicated. For example, KardiaMobile EKG monitors offer users an opportunity to get medical grade EKG readings in under a minute, synced with a smart phone the EKG can be shared. The device is FDA cleared to detect Atrial Fibrillation, Bradycardia, and Tachycardia—the message of either normal EKG or abnormality detected by the device could be sent to the server computer 14s as part of pre-visit patient profile. This process could be done for smart scales, the information can be entered into the system and used for accurate calculations of contrast dosing, etc.

As the patient data is being gathered, it needs to be interpreted within the context of the patient's exam. For instance, if the patient is having a routine brain MR scan, the contents of his lunch are likely meaningless; however, if the patient is having a liver MRI scan measuring the hepatic fat fraction, then the contents and timings of patient's lunch are extremely meaningful and important. All information could be gathered as part of the patient profile, but only relevant data could be used to drive action. The outputs of the interpretation module may include warning flags to be displayed for an operations manager or expert user, suggesting that based on the gathered health data, the patient may not be following prep instructions, the patient may have difficulty completing the exam, etc. The initial models could be based on simple logic. However, as patients begin flowing through the ROCC system 1 and information regarding patient-related delays starts emerging, more sophisticated models (first logistic regression) can be trained to predict delays based on patient characteristics gathered through personal devices. Table 1 (below) shows an example of the patient data correlated with the context of the patient's exam.

TABLE 1

| Contrast | Yes | |
|---|---|---|
| Breath holds | Yes | |
| Prior within 6 months | No | |
| Gender | Male | |
| Age | 55 | years |
| Walking speed | 1.5 | mi/hr |
| Heart rate | 70 | beats/min |
| Respiratory rate | 21 | breaths/min |

| | |
|---|---|
| Weight/height | 235 lbs/5′7″ |
| Recent falls | No |
| Recent blood work | No |
| Contrast | Yes |
| Diabetes | Yes |
| Implant | No |

Some examples of the correlation can include, for example, “Delays with protocol completion likely—YES,” “wide bore—PREFERRED,” “need blood work—YES”, and so forth. Based on this information, a simple logic model may conclude that due to elevated breathing rate (>20 breaths/min), the patient may have trouble with breath holds. Additionally, based on the presented weight and height, the patient is likely to require a wide bore scanner and requires blood work for kidney function check before exam.

The information from the patient monitoring devices 40 can be combined with the patient's history to provide the likelihood of workflow disruption based on a pattern of behavior. For example, if the patient had a prior history of non-compliance, tardiness, or anxiety, this information combined with “real-time” data signals could point to impending difficulties.

Once it is established that there is a higher likelihood of a disruptive exam, proactive measures could be suggested to mitigate the effect. For instance, if the patient had difficulty completing a scan during a previous exam, is exhibiting anxiety based on his health data, there is a higher likelihood the patient may struggle and the exam may take longer, affecting the overall workflow. In the example above, the knowledge that a patient is likely to struggle with breath holds can also drive action. The patient might be asked to come in earlier or may be sent reminders to complete his blood work. The appropriate imaging device 2 can be allocated based on patient needs and an alternate protocol with faster sequences might be used to eliminate any need for breath holds. There are a limited number of levers that an imaging facility may use to accommodate patient needs and/or improve odds of smooth exam. Such a logic-based mapping could be used to provide an appropriate response to potential difficulties.

In some examples, a patient might be asked to come in earlier in cases of prior tardiness, missing blood work, frailty, slow walking speeds, etc. Faster protocols might be suggested in cases of claustrophobia, breathing problems, post-op pain, prior record of excessive patient motion, etc. A choice of the imaging device 2 (e.g., wide bore, 1.5T scanner, etc.) might be dictated by presence of implants, patient weight, claustrophobia, etc. The patient's exam might be rescheduled to a different day if a patient fails to comply with prep instructions, has high anxiety (poor sleep, high heart rate, etc.), is sick, and so forth.

In addition, some of the patient profile information obtained from the patient monitoring devices 40 could be of interest to radiologists in interpreting an exam.

In some embodiments, the ROCC system 1 is configured to track whether a patient is complying with the prep instructions. Some medical exams require fasting. If the patient had a big lunch prior to his scan, the exam may need to be rescheduled. In another example, the patient may have trouble with breath hold sequences and there might be inconsistency in follow-up especially for abdominal exams due to shifted organs after a large meal.

In some embodiments, a detected irregular EEG or EKG may affect gating during scan acquisition and might be meaningful for interpretation.

In some embodiments, blood pressure monitoring may be used to assess patient's level of anxiety. If the patient is anxious about the scan, a referring physician may need to prescribe a sedative. This has to be done prior to the patient's arrival to the imaging facility and can significantly case some of the workflow difficulties.

In some embodiments, high levels of activity may compromise metabolic imaging studies.

In some embodiments, PET and mammography exams may need to be rescheduled or the radiologist may need to be made aware to ensure correct interpretation during a menstrual cycle of a patient.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a method of preparing for an upcoming medical imaging examination, the method comprising:
receiving information about the upcoming medical imaging examination;
receiving patient data from a patient monitoring device associated with a patient who is to undergo the upcoming medical imaging examination;
analyzing the received patient data to determine one or more indicators of a potential issue with the upcoming medical imaging examination based on a comparison of the received patient data with the information about the upcoming medical imaging examination; and
outputting a warning indicative of the determined one or more indicators of the issue with the medical imaging examination.

2. The non-transitory computer readable medium of claim 1, wherein receiving patient data from a patient monitoring device associated with the patient who is to undergo the upcoming medical imaging examination includes:
receiving a user input indicative of the patient providing consent for collecting the patient data from the patient monitoring device; and
conditional on the patient providing the consent, establishing a wireless data connection from the patient monitoring device to a medical information technology (IT) system over which connection the patient data is received.

3. The non-transitory computer readable medium of claim 1, further storing instructions executable by at least one electronic processor to perform an operating method for operating the patient monitoring device, the operating method comprising:
collecting the patient data using the patient monitoring device;
storing the patient data on the patient monitoring device and/or transmitting the patient data to an electronic medical record of the patient who is to undergo the upcoming medical imaging examination; and
transmitting the patient data to a medical information technology (IT) system that performs the analyzing.

4. The non-transitory computer readable medium of claim 1, wherein the information about the upcoming medical imaging examination includes one or more of fasting requirements, patient demographic information, and any available information on chronic conditions of the patient.

5. The non-transitory computer readable medium of claim 1, wherein the patient data includes one or more of a presence of an implant in the patient, a use of a walking assistance device by the patient, and one or more vital signs of the patient.

6. The non-transitory computer readable medium of claim 1, wherein the patient data includes at least pedometer data indicative of a mobility status of the patient, and the warning includes an indication that a wheelchair or other walking assistance device is available for the patient for the medical imaging examination.

7. The non-transitory computer readable medium of claim 1, wherein the patient data includes at least patient breath data indicative of whether the patient has consumed food during a require fasting period before the medical imaging examination, and the warning includes an indication that a radiologist should account for the food consumption by the patient.

8. The non-transitory computer readable medium of claim 1, wherein the patient data includes at least a presence of an implant in the patient, and the warning includes an indication that a radiologist should account for the implant presence during the medical imaging examination.

9. The non-transitory computer readable medium of claim 1, wherein the patient data includes one or more of cardiac and blood pressure data of the patient, and the warning includes an indication to determine whether the patient should be administered a sedative prior to the medical imaging examination.

10. The non-transitory computer readable medium of claim 1, wherein the patient data includes patient over-exertion, and the warning includes an indication to determine whether the patient should rest prior to the medical imaging examination or whether to reschedule the medical imaging examination.

11. The non-transitory computer readable medium of claim 1, wherein the patient data includes cardiac data of the patient, and the warning includes an indication to account for a cardiac arrhythmia or other irregular cardiac rhythm during interpretation of the medical imaging examination.

12. A system for preparing for an upcoming medical examination, the system comprising:
a patient monitoring device associated with a patient who is to undergo the upcoming medical imaging examination; and
at least one electronic processor programmed to perform a method of preparing for the upcoming medical imaging examination, the method comprising:
receiving information about the upcoming medical imaging examination;
providing a user interface (UI) on the patient monitoring device;
receiving an input on the UI indicative of a consent of the patient to transmit patient data from the patient monitoring device;
analyzing the received patient data based on information about the upcoming medical imaging examination to determine one or more indicators of a potential issue with the upcoming medical imaging examination; and
outputting a warning indicative of the determined one or more indicators of the issue with the medical imaging examination.

13. The system of claim 12, wherein receiving patient data from a patient monitoring device associated with the patient who is to undergo the upcoming medical imaging examination includes:
conditional on the patient providing the consent, establishing a wireless data connection from the patient monitoring device to a medical information technology (IT) system over which connection the patient data is received.

14. The system of claim 12, wherein the at least one electronic processor is further programmed to perform an operating method for operating the patient monitoring device, the operating method comprising:
collecting the patient data using the patient monitoring device;
storing the patient data on the patient monitoring device and/or transmitting the patient data to an electronic medical record of the patient who is to undergo the upcoming medical imaging examination; and
transmitting the patient data to a medical information technology (IT) system that performs the analyzing.

15. The system of claim 12, wherein the information about the upcoming medical imaging examination includes one or more of fasting requirements, patient demographic information, and information on at least one chronic condition of the patient.

16. The system of claim 12, wherein the patient data includes one or more of a presence of an implant in the patient, a use of a walking assistance device by the patient, and one or more vital signs of the patient.

17. A method of preparing for an upcoming medical imaging examination, the method comprising:
acquiring patient data using a patient monitoring device connected with a patient who is to undergo the upcoming medical imaging examination;
transmitting the patient data to a medical information technology (IT) system;
using the medical IT system, analyzing the patient data using a context of the upcoming medical imaging examination stored at the medical IT system to determine one or more indicators of a potential issue with the upcoming medical imaging examination; and
outputting, on a user interface operatively connected with the medical IT system, a warning indicative of the determined one or more indicators of the issue with the medical imaging examination.

18. The method of claim 17, further comprising:
at the patient monitoring device, analyzing the patient data to determine one or more physical fitness metrics; and
displaying the one or more physical fitness metrics at the patient monitoring device.

19. The method of claim 17, wherein acquiring patient data using a patient monitoring device connected with a patient who is to undergo the upcoming medical imaging examination includes:
receiving a user input indicative of the patient providing consent for collecting the patient data from the patient monitoring device; and
conditional on the patient providing the consent, establishing a wireless data connection from the patient monitoring device to the medical IT system.

20. The method of claim 17, further comprising:
storing the patient data on the patient monitoring device and/or transmitting the patient data to an electronic medical record of the patient who is to undergo the upcoming medical imaging examination; and
transmitting the patient data to the medical IT system.
